(19) United States
(12) Patent Application Publication
(10) Pub. No.: US 2011/0196035 A1
(43) Pub. Date: Aug. 11, 2011

(54) Temporally-Controlled Treatment of Joint Disease

(76) Inventors: Oleg V. Kolomytkin, Bossier City, LA (US); Andrew A. Marino, Belcher, LA (US)

(21) Appl. No.: 13/013,904

(22) Filed: Jan. 26, 2011

Related U.S. Application Data
(60) Provisional application No. 61/337,461, filed on Feb. 5, 2010.

(51) Int. Cl. A61K 31/277; A61P 19/02; A61P 29/00

(52) U.S. Cl. 514/523

(57) ABSTRACT

A composition and method for treatment of osteoarthritis is disclosed. The composition includes at least one soluble injectable biocompatible carrier having a viscosity within prescribed limits and an amount of a calcium-channel blocker chosen in relation to the viscosity of the carrier. The concentration of the calcium-channel blocker in the joint synovial fluid is controlled according to the invention whereby effective treatment of osteoarthritis is provided.

### RELATED APPLICATION

This application claims priority to U.S. Ser. No. 61/337,461 filed Feb. 5, 2010, hereby incorporated by reference in its entirety.

### FIELD OF INVENTION

The present invention relates to the treatment of pain and inflammation in body tissues, including the synovial cells of body joints. In particular, the present invention relates to methods and compositions for treating osteoarthritis using compositions composed of a calcium-channel blocker dissolved in a viscous carrier.

### BACKGROUND

Osteoarthritis is a joint disease in which the cartilage that covers the ends of the bones that form the joint is degraded by the action of various enzymes including matrix metalloproteinases (MMPs) that are secreted into the synovial fluid of the joint by synovial cells in response to stimulation by proinflammatory cytokines, resulting in pain, swelling, loss of joint cartilage, and abnormal restriction of joint motion. Calcium channels are glycoprotein structures located in the membrane of cells, including synovial cells, that allow calcium ions to pass into a cell during the process of synthesis and secretion of MMPs. Calcium-channel blockers are chemical agents capable of blocking the entry of calcium ions into cells.

Calcium-channel blockers may be useful to treat osteoarthritis. However direct injection of an aqueous solution of a blocker into a joint may produce undesirably high initial concentrations of the blocker in the joint synovial fluid which may be harmful to the synovial cells, and there is also the danger that the blocker may be rapidly removed from the joint by means of natural physiological processes before the blocker accomplishes its intended purpose of relieving pain and inflammation. Calcium-channel blockers may also be used in controlled-release formulation for treatment of osteoarthritis in the expectation that prolonged presence of the drug will have the desired effect. But all presently known methods for producing controlled release of drugs into joint synovial fluid have certain drawbacks and limitations that impair effectiveness.

The problem of exposing synovial cells in an osteoarthritic joint to a safe and effective concentration of a calcium-channel blocker in the joint synovial fluid is solved by dissolving the blocker in an injectable viscous biocompatible carrier that is soluble in synovial fluid.

### SUMMARY

According to its major aspects and broadly stated, the present invention provides a composition and method for treating joint pain, inflammation and loss of function associated with osteoarthritis by controlling the amount of certain agents that can block the movement of calcium ions from synovial fluid into synovial cells through structures embedded in the cell membrane called ion channels. Such agents are referred to herein as “calcium-channel blockers”.

The calcium-channel blocker is preferably a phenylalkylamine, a dihydropyridine, or a benzothiazepine. Preferably the calcium-channel blocker will be administered as a combination of enantiomers for those compounds having more than one enantiomer. The use of mixed enantiomers in the present invention is necessary for achieving a therapeutic effect. In particularly preferred embodiments, the calcium-channel blocker is verapamil, nicardipine, or diltiazem. In a further preferred embodiment, the calcium-channel blocker is verapamil consisting of equal parts of its two enantiomers.

One embodiment of the present invention comprises a method for treating osteoarthritis, which comprises injecting into the closed cavity of the joint a safe and effective amount of a calcium-channel blocker in a soluble viscous biocompatible carrier.

The method of the present invention treats the underlying cellular processes that lead to the pain and tissue destruction associated with osteoarthritis. In one embodiment, the invention comprises administering to the synovial tissue a safe and effective amount of calcium-channel blocker; that is, an amount sufficient to reduce any or all of the symptoms of osteoarthritis without producing any unacceptable side-effects including but not limited to cell death, injury, and joint swelling. The present invention provides a means for interfering with cell signaling by the cytokine interleukin-1 (IL-1), the major inflammatory cytokine associated with osteoarthritis, thereby leading to lower MMP levels and correspondingly lower cartilage destruction and resultant pain. It is believed that this method does not necessarily affect the production of IL-1, but rather alters its consequences by interfering with the synthesis of MMPs at a point subsequent to the binding of IL-1 to its receptor on the surface of the cells, which is known to those skilled in the art as being an early critical step in the process of MMP synthesis.

In its various embodiments, the present invention provides several treatment modalities to users. Treatment may consist of the administration of a safe and effective amount of the composition. Alternatively, treatment may include administration of the composition in combination with administration of one or more other osteoarthritis treatment agents. The treatment can readily be customized to the individual patient's needs, and may be used instead of or in conjunction with other treatment modalities including, but not limited to, physical therapy, treatments that provide localized pain relief (heat, massage, application of liniments, etc.), and other medications that help reduce disability, relieve pain, and improve the patient's quality of life.

Other features and advantages of the present invention will be apparent to those skilled in the art from a careful reading of the Detailed Description of the invention presented below.

### BRIEF DESCRIPTION OF DRAWINGS

FIG. 1 shows an experimental set-up for determining the time-dependent concentration of a calcium-channel blocker in synovial fluid that occurs following insertion of a test composition into a joint.

FIG. 2 shows the time-dependent changes in racemic verapamil concentration produced by a test composition having a concentration-to-viscosity ratio (CVR) of 1.5 mg/mL/mPa·s.

FIG. 3A shows the effect of various concentrations of racemic verapamil on the inhibition of matrix metalloproteinase (MMP) production by synovial cells for exposure durations between 10 and 100 hours.

FIG. 3B shows the effect of various durations of exposure of synovial cells to racemic verapamil on the inhibition of matrix metalloproteinase (MMP) production by synovial cells for concentrations of verapamil between 0.019-0.050 mg/mL.

FIG. 4 shows the time-dependent changes in racemic verapamil concentration produced by a test composition having a concentration-to-viscosity ratio (CVR) of 0.15 mg/mL/mPa·s.

FIG. 5, curve A, shows the time-dependent change in racemic verapamil concentration produced by a test composition having a concentration-to-viscosity ratio (CVR) of 0.015 mg/mL/mPa·s. Curve B shows the change in verapamil concentration produced by a test composition with a concentration-to-viscosity ratio (CVR) of 0.005 mg/mL/mPa·s.

FIG. 6 shows the time-dependent changes in racemic verapamil concentration produced by a test composition having a concentration-to-viscosity ratio (CVR) of 0.04 mg/mL/mPa·s.

FIG. 7 shows the time-dependent changes in racemic verapamil concentration produced by a test composition having a concentration-to-viscosity ratio (CVR) of 3.0 mg/mL/mPa·s.

FIG. 8 shows the microscopic appearance of synovial tissue in the knee joint of a mouse treated according to the invention using nifedipine at a concentration-to-viscosity ratio (CVR) of 0.028 mg/mL/mPa·s.

FIG. 9 shows the therapeutic results obtained in patient R.S., a 43-year-old male with osteoarthritis of the right knee, following treatment according to the invention using a racemic verapamil at a concentration-to-viscosity ratio (CVR) of 0.0015 mg/mL/mPa·s.

FIG. 10 shows the therapeutic results obtained in patient T.J., a 46-year-old male with osteoarthritis of the right knee, following treatment according to the invention using a racemic verapamil at a concentration-to-viscosity ratio (CVR) of 0.0015 mg/mL/mPa·s.

FIG. 11 shows the therapeutic results obtained in patient C.E., a 64-year-old male with osteoarthritis of the left knee, following treatment according to the invention using a racemic verapamil at a concentration-to-viscosity ratio (CVR) of 0.0015 mg/mL/mPa·s.

### DETAILED DESCRIPTION

This disclosure is directed generally to injectable compositions that include a calcium-channel blocker dissolved in a soluble viscous carrier. In certain preferred embodiments, the concentration of the blocker and the viscosity of the carrier may be selected to treat the inflammation, pain, and tissue destruction associated with osteoarthritis.

Without intending to be bound by theory, it is believed that the presence of proinflammatory cytokines in the joints, for example knee joints, leads to the entry of certain ions into synovial cells and other cells in the joint, and to other cellular events including activation of protein kinase C and changes in intercellular communications, ultimately resulting in secretion by the cells of various proteins; the overall process is referred to herein as “cell signalling.” If cell signalling is not regulated properly, osteoarthritis may occur as characterized by symptoms that include pain, inflammation, abnormal neovascularization, bone and cartilage erosion, an abnormal restriction of joint motion.

Entry of certain ions, for example calcium ions, has been found to be critically important to the ability of synovial cells to secrete MMPs, which are believed to be primarily responsible for the destruction of joint cartilage that leads to joint pain associated with osteoarthritis. Interleukin-one is an important proinflammatory cytokine in the cell signalling process leading to secretion of MMPs into the joint synovial fluid. See, “Biochemistry and Metabolism of Articular Cartilage in Osteoarthritis,” H. J. Mankin and K. D. Brant, in Osteoarthritis: Diagnosis and Medical/Surgical Management, 2^{nd} Ed., R. W. Moskowitz, D. S. Howell, V. M. Goldberg, and H. J. Mankin, W.B. Saunders Co., Philadelphia (1992), which is incorporated herein by reference.

The specific MMPS whose levels in joint fluid are regulated by IL-1, and whose dysregulation may mediate development of osteoarthritis, include MMP-1, also known as collagenase-1; MMP-2, also known as gelatinase A; MMP-3, also known as stromelysin-1; MMP 8 and also known as collagenase-2; and MMP-13, also known as collagenase-3. The levels of MMP activity produced by synovial tissue from patients having osteoarthritis is greater than the corresponding levels obtained from patients who do not have arthritis (see “Increased Intercellular Communication Through Gap Junctions May Contribute to Progression of Osteoarthritis,” A. A. Marino, D. D. Waddell, O. V. Kolomytkin, W. D. Meek., R. Wolf, K. K. Sadasivan, and J. A. Albright; Clinical Orthopaedics & Related Research 422:224-232 (2004), which is incorporated herein by reference.

Osteoarthritis is believed to be related to the level of regulation of the calcium channels in the sense that the disease develops when an inappropriate amount of calcium ions pass through calcium channels during cell signalling. So it may be said that the present invention treats osteoarthritis by allowing fewer ions to pass through the membrane which results in a situation that is closer to normal. The process by which fewer calcium ions pass through a calcium channel is herein termed “blocking” and, as mentioned previously, a chemical agent that produces or causes such blocking is herein termed a “calcium-channel blocker.” Representative examples of calcium-channel blockers include verapamil, nifedipine, diltiazem, amlodipine, bepridil, felodipine, gallopamil, isradipine, nicardipine, nimodipine, nitrendipine, and mixtures thereof. The use of any of these terms to describe a blocker is meant to include both the compound in its free base form and those forms that include a pharmaceutically acceptable salt. A detailed list of the calcium-channel blockers useful in the present invention is given in Mak, U.S. Pat. No. 6,190,691. It is contemplated that all known and future discovered calcium-channel blockers will be useful in the present invention.

Some calcium-channel blockers can occur in two mirror-image stereoscopic forms; verapamil is an example, and diltiazem is another example. The mirror image forms are herein termed “enantiomers.” Enantiomers can be termed “(+)-” or “(−)-” depending upon whether they rotate polarized light in a clockwise or counter-clockwise direction. For example, (+)-verapamil rotates light in a clockwise direction, and (−)-verapamil rotates light in a counter-clockwise direction. The biological effects of different enantiomers commonly differ. When approximately equal amounts of enantiomeric molecules are mixed together, the product is herein referred to as a “racemic mixture.” A mixture of approximately equal percentages of (+)-verapamil and (−)-verapamil is referred to herein as “racemic verapamil.”

Calcium-channel blockers have been found to be capable of interfering with the effect of IL-1 on synovial cells (see “Interleukin 1β Switches Electrophysiological States of Synovial Fibroblasts”; O. V. Kolomytkin, A. A. Marino, K. K. Sadasivan, R. E. Wolf, and J. A. Albright, American Journal of Physiology, 273 (Regulatory Integrative Comp. Physiol. 42):R1822—R1828 (1997), which is incorporated herein by reference. Without intending to be bound by theory, it is believed that the cellular mechanism responsible for the therapeutic effects of treating osteoarthritis with calcium-channel blockers in accordance with the present invention involves their ability to antagonize the proinflammatory effect of IL-1 which otherwise would lead to elevated levels of MMPs resulting in the chronic inflammation, cartilage destruction, pain, and reduced joint mobility associated with osteoarthritis. According to the present invention the above-described antagonization is accomplished for an optimal period of time without producing undesirable effects on the viability or metabolism of the synovial cells. For example, by reducing the entry of calcium ions into synovial cells for a predetermined period, under appropriate conditions of safety, the calcium signalling pathway is altered, thereby preventing intracellular events that would otherwise culminate in inflammation and cartilage destruction.

Calcium-channel blockers are commonly used for treating a variety of cardiac conditions, including atrial fibrillation, supraventricular tachycardias, hypertrophic cardiomyopathy and hypertension, migraine headaches, prevention of brain damage, and other disorders. The typical mode of administration of calcium-channel blockers is by mouth.

Calcium-channel blockers injected into a joint may interfere with the effect of IL-1 on synovial cells, and may be useful to treat osteoarthritis. See for example, U.S. Pat. No. 7,767,710, U.S. patent application Ser. No. 11/138,738 (abandoned) and pending U.S. patent application Ser. No. 12/238,908, which teach that up to 2.0 mg of a blocker may be injected into the joint to treat osteoarthritis, and Mak which teaches that 20-40 mg of (+)-verapamil may be injected to treat rheumatoid arthritis.

The use of calcium-channel blockers as taught in the above-mentioned patent applications is sometimes ineffective in treating osteoarthritis for one or more reasons including: the unavailability of a sufficient concentration of the blocker in the joint synovial fluid; the presence of the blocker in said fluid for too short a period of time before the blocker is removed from the joint by natural physiological processes; the absence of the blocker enantiomer whose presence is critical for the production of the desired effect; excessively high levels of the blocker in joint synovial fluid resulting in adverse effects on joint synovial tissue; in cases where the blocker is administered by mouth, excessively high levels of the blocker in the stomach, intestines, and blood, resulting in adverse effect on these tissues. Methods and compositions are therefore needed to permit delivery of calcium-channel blockers to synovial cells via the synovial fluid more effectively than is taught in the present art.

The problem of exposing synovial cells in an osteoarthritic joint to an amount of a calcium-channel blocker that may produce a safe and effective treatment substantially greater than that obtainable using the present art has been solved by dissolving the calcium-channel blocker in an injectable soluble viscous biocompatible carrier. The concentration of the calcium-channel blocker and the viscosity of the carrier are chosen in relation to one another within prescribed limits, whereby a precise level of treatment may be obtained for a specific time interval as required in the particular case.

As used herein, the term “biocompatible” means a carrier that has no medically unacceptable toxic or injurious effects on biological function; the term “viscous biocompatible carrier” means a biocompatible carrier having a viscosity greater than that of saline at 37° C., which is known by those skilled in the art to be about 0.7 millipascal·seconds (mPa·s); the term “soluble viscous biocompatible carrier” means a viscous biocompatible carrier that dissolves in joint synovial fluid.

In distinction to prior teaching regarding compositions injected into a joint, in the present invention the viscosity of the composition is regulated statically and dynamically or temporally, the two terms meaning the same thing and used herein interchangeably, by means of which the concentration of the calcium-channel blocker in the synovial fluid is controlled. The term “static viscosity” is used herein to refer to the viscosity of the composition prior to injection. Without intending to be bound by theory, it is believed that, following intra-articular injection of a composition, its static viscosity will decrease continuously because of the movement of water molecules from the synovial fluid into the composition, which process occurs simultaneously with and is coupled to the movement of blocker molecules into the synovial fluid from the composition, whereupon they are available to interact with the synovial cells to produce the desired changes in cell signalling. It may therefore be said that, following injection, the viscosity of the composition is regulated temporally, and that said temporal regulation of the viscosity is a critical factor in releasing the blocker molecules into the synovial fluid.

It is recognized that the rate at which blocker molecules pass into the synovial fluid depends on the static viscosity of the composition and on the particular point in time following injection of the composition into the joint that the rate determination is made. For example, calcium-channel blocker molecules in a composition having a static viscosity of 20 mPa·s will move into the synovial fluid more quickly compared with molecules in a composition having a static viscosity of 200 mPa·s. As a further example, assuming that a composition has a static viscosity of 200 mPa·s, the rate of movement of blocker molecules from the composition into the synovial fluid occurring 20 hours after intra-articular injection will differ from the corresponding rate occurring 50 hours after intra-articular injection.

It is further recognized that the actual concentration of blocker molecules in the synovial fluid will depend not only on the static viscosity of the composition and the release of the calcium-channel blocker into the synovial fluid as a consequence of the dynamic viscosity changes of the composition, but also on the rate at which the calcium-channel blocker is removed from the synovial fluid by natural physiological processes. If the static viscosity is too low, diffusion of the calcium-channel blocker into the synovial fluid may occur too rapidly, whereupon it will be removed from the joint by means of natural physiological processes with the result that the blocker will have been present in the synovial fluid for too brief a time to produce the desired effect on signalling in the synovial cells. For example, if the static viscosity is about 3 mPa·s and 1 mL of a composition containing 5 mg/mL of a calcium-channel blocker is injected into a joint containing 10 mL of synovial fluid, the calcium-channel blocker is cleared from the joint in about 50 hours. However, if the static viscosity is about 350 mPa·s, when 1 mL of a composition containing 5 mg/mL of a calcium-channel blocker is injected into a joint containing 10 mL of synovial fluid, the calcium-channel blocker remains in the joint for about 125 hours, whereby it may exert the desired effect on signalling in synovial cells for a longer period of time.

If the static viscosity of the composition is too high, diffusion of the calcium-channel blocker into the synovial fluid may occur too slowly, whereupon it will not be removed from the joint by means of natural physiological processes resulting in exposure of the synovial cells to the blocker for an excessively long timer period, which may cause adverse effects on the synovial cells. For example, if the static viscosity is about 5000 mPa·s and 1 mL of a composition containing 15 mg/mL of a calcium-channel blocker is injected into a joint containing 10 mL of synovial fluid, the blocker will not be cleared from the joint until more than 600 hours have elapsed since the injection.

According to the invention, the static viscosity must be chosen in relation to the choice of the concentration of the calcium-channel blocker to avoid dangerously high and dangerously persistent concentrations of the calcium-channel blocker in the joint synovial fluid. For example, if the static viscosity is about 10 mPa·s and 1 mL of a composition containing 30 mg of verapamil is injected into a joint containing 10 mL of synovial fluid, the blocker concentration in said fluid will reach 0.14 mg/mL about 15 hours after injection, thereby resulting in the death of some synovial cells.

In certain preferred embodiments the static viscosity and the amount of calcium-channel blocker may be chosen in relation to one another so that the temporal changes in the viscosity of the composition that occur following its intra-articular injection result in the presence of the desired amount of calcium-channel blocker in the synovial fluid for the desired time interval.

It may therefore be said that according to the present invention, following intra-articular injection, the concentration of a calcium-channel blocker in the synovial fluid and the duration of its presence therein prior to removal by natural physiological processes may be controlled by controlling the static viscosity of the composition and the amount of calcium-channel blocker it contains, both steps being taken in relation to one another and within particular limits, with the result that the concentration of the blocker may remain above the threshold for affecting MMP production for a period sufficient to produce a desired effect on cell signalling while at the same time providing that the blocker is not present in the joint at too high a concentration or for too long a period time that may result in injurious or lethal effects on the synovial cells.

In some embodiments, it is contemplated that the compositions in accordance with the present invention will be significantly more effective in reducing pain and improving function in a diseased joint than would use of the calcium-channel blocker alone, that is, in the absence of a soluble viscous carrier.

The term “safe and effective treatment” means a cure, mitigation, treatment or prevention of disease in a human being at an acceptable risk/benefit ratio caused by the application of physical or chemical agents, or the production or initiation of a chemical or physical change in a model system designed to represent biochemical processes occurring in the human body wherein the change, were it to occur in the body, would be regarded as a cure, mitigation, or treatment of a disease, and wherein any adverse biological changes that may occur in the model system are deemed acceptable.

The term “safe and effective composition” means a composition that may produce safe and effective treatment. The amounts of the components in a safe and effective composition will depend on various factors including but not limited to the nature and severity of the disease of the subject being treated, the weight and age of the subject, and the frequency of injection of the composition. For example, certain compositions of the present invention may be administered to produce a therapeutic effect applicable to the treatment of osteoarthritis.

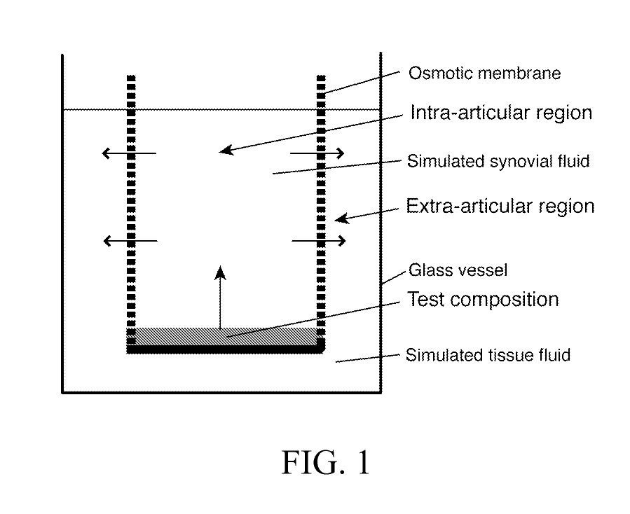

An apparatus suitable for determining the preferred embodiments of the present invention is shown in FIG. 1 which depicts the experimental set-up used to measure the concentration of a calcium-channel blocker in simulated joint synovial fluid inside a simulated joint following insertion of a test composition into said joint.

The term “test composition” is used herein to refer to any specific embodiment of the invention whose behavior is being evaluated in the experimental set-up.

The term “simulated synovial fluid” is used herein to refer to an aqueous solution containing the same ionic composition, pH, and albumin concentration as normal human synovial fluid.

The term “half-life” is used herein to refer to the time interval within which half of any particular constituent or component of joint synovial fluid is removed by natural physiological processes, whether or not said constituent or component is replaced by like kind. For example, the albumin present in a human knee joint, which is continuously being removed and replaced by newly synthesized albumin, has a half-life of thirteen hours, more or less (see Owen, S. G., Francis, H. W. and Roberts, M. S. Disappearance kinetics of solutes from synovial fluid after intra-articular injection. Br. J. Clin. Pharmac. 38:349-355, 1994, which is incorporated herein by reference). The net result of the process is that the albumin concentration in the joint remains within physiological limits even though about half the albumin molecules in the synovial fluid at any particular time were not present thirteen hours earlier. It is known to those skilled in the art that a calcium-channel blocker released from a test composition into the synovial fluid will be nonspecifically bound to the albumin, and therefore that the half-life of the calcium-channel blocker in the simulated joint fluid will be thirteen hours, more or less.

The term “osmotic membrane” is used herein to refer to a cylindrical cellulose membrane having a sufficient number of pores to allow about half of the albumin to pass from the intra-articular to the extra-articular region of the simulated knee joint depicted in FIG. 1 with a half-life of thirteen hours, more or less.

It will be recognized by those skilled in the art that when a test composition consisting of a calcium-channel blocker and a soluble viscous carrier is placed intra-articularly in the simulated knee joint, that is, inside the osmotic membrane, the viscous carrier will immediately begin dissolving in the synovial fluid, by which is meant that water molecules from the synovial fluid will move into the composition and blocker molecules will move into the synovial fluid from the composition, whereupon they will become bound to albumin and will therefore become available to interact with synovial cells were they present. The surrogate process in the experimental set-up representing the removal of the blocker from the synovial fluid that begins following injection of a composition into the joint is the passage of the blocker/albumin complex through the pores in the osmotic membrane.

The apparatus depicted in FIG. 1 was used to identify the preferred embodiment of the invention. This was accomplished by systematically varying the components of the test composition and, for each test composition, measuring the concentration of the calcium-channel blocker in the synovial fluid as a function of time. After having made these measurements at various times following insertion of the test composition into the simulated joint, the effect on MMP production by synovial cells for relevant blocker concentrations and times of exposure was measured employing a confluent layer of HIG-82 synovial cells in serumless Neuman-Tytell growth medium, which is a model system commonly used by those skilled in the art to represent human synovial tissue for the purpose of studying the proinflammatory effects of IL-1 and the antagonization thereon produced by agents of interest, for example, calcium-channel blockers.

The MMP levels in the medium conditioned by the cells were measured using the assay described in Kolomytkin, O. V., Marino, A. A., Waddell, D. D., Mathis, J. M., Wolf, R. E., Sadasivan, K. K. and Albright, J. A. IL-1β-induced Production of Metalloproteinases by Synovial Cells Depends on Gap-junction Conductance. Am. J. Physiol. Cell Physiol. 282:C1254-C1260, 2002, which is incorporated herein by reference and should be consulted for more detail.

We made the surprising discovery that when the concentration of the calcium-channel blocker is chosen in relation to the viscosity of the carrier, the rate of diffusion of the blocker into the synovial fluid may be balanced in relation to the rate of removal of the blocker by natural physiological processes, whereby the concentration of the blocker in the synovial fluid may be temporally controlled as desired to produce a safe and effective treatment. Said relation may be expressed as a ratio of the concentration of the calcium-channel blocker expressed in units of mg/mL to the viscosity of the carrier expressed in units of mPa·s, referred to herein as the “concentration-viscosity ratio” or the “CVR.” For example, when the test composition contains 15 mg of a racemic mixture of verapamil in one milliliter of a carrier having a viscosity of 10 mPa·s, the corresponding CVR is 1.5 mg/mL/mPa·s.

When the test composition depicted in FIG. 1 was 15 mg of a racemic mixture of verapamil in one milliliter of carrier having a viscosity of 10 mPa·s, corresponding to a CVR of 1.5 mg/mL/mPa·s, the verapamil concentration in the joint synovial fluid was found to be greater than 0.05 mg/mL for 34 hours, which was a period of time sufficient to result in a 61% reduction of MMP production in the standard assay. Additionally, the verapamil was not cleared from the joint until about 100 hours had elapsed from the time the composition was inserted into the joint. During the 100-hour interval the peak concentration was 0.069 mg/mL and the average concentration was 0.033 mg/mL. Exposure of synovial cells in the standard assay to 0.033 mg/mL for 100 hours resulted in a 31% reduction of MMP production in the standard assay. It will thus be clear to those skilled in the art that the invention produces two related but distinct effects, one effect associated with the duration of time that the concentration of the blocker in the joint synovial fluid was above 0.05 mg/mL, and a second effect associated with the average value of the concentration of the blocker during the time it remained in the joint. It will be clear to one skilled in the art which combination of effects are desirable in a particular case to produce safe and effective treatment.

As a further example, when the composition was 15 mg of a racemic mixture of verapamil in one milliliter of a carrier having a viscosity of 100 mPa·s, corresponding to a CVR of 0.15 mg/mUmPa·s, the verapamil composition in the joint synovial fluid remained above 0.05 mg/mL for about 32 hours, which was a period of time sufficient to result in a 59% reduction of MMP production in the standard assay. The verapamil was not cleared from the joint until more than 190 hours had elapsed, during which time interval the average concentration was 0.024 mg/mL. Exposure of synovial cells in the standard assay to 0.024 mg/mL for 190 hours resulted in a 46% reduction of MMP activity in the standard assay.

In some embodiments the blocker concentration in the joint synovial fluid may be further lengthened, as desired. For example, when the composition contained 5 mg of a racemic mixture of verapamil in one milliliter of a carrier having a viscosity of 1000 mPa·s, which corresponds to a CVR of 0.005 mg/mL/mPa·s, the verapamil was not cleared from the joint until more than 294 hours had elapsed during which time interval the average concentration was 0.011 mg/mL. Exposure of synovial cells in the standard assay to 0.011 mg/mL for 294 hours resulted in a 61% reduction of MMP activity in the standard assay.

The trypan blue exclusion test is commonly used to evaluate whether cells are alive or dead. The test consists of adding an appropriate amount of trypan blue dye to the environment of the cells. Cells are able to exclude the dye if they are healthy, but if they are injured or dead the dye enters the cells and stains them blue. By means of the trypan blue exclusion test, harmful or lethal effects of certain levels of calcium-channel blockers on cells can be determined.

We made the further surprising discovery that the therapeutic effect produced by the invention is substantially diminished or lost when the synovial cells are treated with a calcium-channel blocker for an excessive period of time. For example, when the composition contained 20 mg of a racemic mixture of verapamil and had a viscosity of 5000 mPa·s, which corresponds to a CVR of 0.0040 mg/mL/mPa·s, the composition of the verapamil in the joint was above 0.05 mg/mL for 235 hours. Exposure of synovial cells in the standard assay under these conditions resulted in an unacceptable level of cell death as assessed using the trypan blue assay. Further similar studies showed that it was necessary to prevent the calcium-channel blocker concentration from remaining at or above 0.05 mg/mL for more than about 100 hours.

We made the further surprising discovery that the therapeutic effect is substantially impaired when synovial cells are treated with a high concentration of a calcium-channel blocker above a particular level. For example, when the composition contains 30 mg of a racemic mixture of verapamil and has a viscosity of 10 mPa·s, which corresponds to a CVR of 3.0 mg/mL/mPa·s, the peak composition of the verapamil in the joint was 0.14 mg/mL. Exposure of synovial cells in the standard assay to this concentration of racemic verapamil for more than about twenty-four hours resulted in an unacceptable level of cell death as assessed using the trypan blue assay. Further similar studies showed that to obviate the occurrence of an unacceptable level of synovial-cell death it was necessary to prevent the calcium-channel blocker concentration from rising above about 0.1 mg/mL.

Without being bound by theory, it is believed that the adverse effects produced by exposure of synovial cells to a calcium-channel blocker for an excessively long period or to an excessively high concentration arise from physiological stress, a process termed herein “metabolic poisoning.”

On the basis of studies similar to those described, the safe and effective range of CVR was determined to be 0.0001-2.0 mg/mL/mPa·s, preferably 0.01-2.0 mg/mUmPa·s, preferably 0.015-1.5 mg/mL/mPa·s. Said studies showed the that effective range of the calcium-channel blocker concentration was about 2 mg/mL to about 30 mg/mL, and effective range of the carrier viscosity was about 2 mPa·s to about 5000 mPa·s.

When the composition contained 10 mg of a (+)-verapamil dissolved in saline as taught in Mak for treating rheumatoid arthritis (CVR=14.3 mg/ml/mPa·s), the verapamil was cleared from the joint in about 101 hours, during which time the average concentration was 0.024 mg/mL. Exposure of synovial cells in the standard assay to 0.024 mg/mL of (+)-verapamil for 101 hours resulted in a percent inhibition of MMP activity of about 8%. Thus the teaching of Mak that (+)-verapamil is effective for treating rheumatoid arthritis does not provide effective treatment of osteoarthritis.

U.S. patent application Ser. No. 11/138,744 teaches that the greatest amount of a calcium-channel blocker dissolved in saline, whose viscosity at 37° C. is known to those skilled in the art to be about 0.7 mPa·s, that can safely be injected into a joint is 2 mg/mL (CVR=2.9 mg/mL/mPa·s). When a composition containing 2 mg of a racemic mixture of verapamil at a viscosity of 0.7 mPa·s, the peak verapamil was only 0.009 mg/mL and the verapamil was cleared from the joint in about 35 hours, during which time the average concentration was 0.006 mg/mL. Exposure of synovial cells in the standard assay under these conditions resulted in a 13% reduction of MMP production in the standard assay. It will be clear to those skilled in the art that all smaller concentrations of verapamil in which saline is the carrier will result in a proportionally smaller average concentration and consequently will be proportionately less therapeutically effective. Thus the effectiveness of the compositions formulated in accordance with the present invention are significantly more effective in treating osteoarthritis than the compositions taught in U.S. patent application Ser. No. 11/138,744.

Soluble viscous carriers suitable for use in the present invention include but are not limited to aqueous solutions containing: glycerol; glycose; lactose; sorbitol; mannitol; polyethylene glycol having a molecular mass between 200-20,000 Daltons; ethyl oleate; ethyl laurate; collagen; gelatin; hyaluronan.

Calcium-channel blockers useful in the compositions of the invention may either be soluble or insoluble in the soluble viscous carrier. For example, verapamil is soluble in glycerol, which is soluble in joint synovial fluid, whereas nifedipine is substantially insoluble in glycerol. In certain embodiments, compositions are provided that comprise use of an excipient whose purpose is to solubilize a calcium-channel blocker in the soluble viscous carrier.

Each excipient must be acceptable in the sense of being compatible with the subject composition and its components and not injurious to the patient. Some examples of materials that may serve as pharmaceutically acceptable solubilizing excipients is given in Strickley, R. G., Solubilizing excipients in oral and injectable formulations; Pharma. Res. 21:201, 2004.

It is understood that a therapeutic composition according to this invention will typically vary depending on the level of disease in the joint, and the type of joint being treated. For example, it is known that knee joints contain differing amounts of synovial fluid, depending on the extent of osteoarthritis in the joint and the size of the joint (Waddell, D. D. and Marino, A. A., Chronic knee effusions in patients with advanced osteoarthritis: implications for functional outcome of viscosupplementation; J. Knee Surg. 2008, which is incorporated by reference herein). For example, when a composition containing a specific amount of calcium-channel blocker is injected directly into a joint, the concentration of calcium-channel blocker in the composition will be diluted by the amount of the synovial fluid in the joint. Accordingly, in an embodiment, the concentration of the calcium-channel blocker in the composition as injected may have to be sufficiently high to elevate the total concentration of ion-channel regulators in the synovial fluid to a therapeutic level. Various factors associated with the affected joint will determine the particular preferred embodiment of the invention.

Other joints in the human body typically have an amount of synovial fluid that is less than the volume of synovial fluid present in the knee joint. Using the principles described above, one skilled in the art may easily determine the preferred embodiment of the invention for the treatment of various levels of osteoarthritis in these other joints.

Methods contemplated herein may further include administration of one or more other agents suitable for the treatment of osteoarthritis. The treatment method of the present invention may readily be customized to the individual patient's needs, and may be used instead of or in conjunction with other treatment modalities including but not limited to physical therapy, joint physical exercise, treatments that provide localized pain relief including but not limited to heat, massage, application of liniments, and with other medications that help reduce disability, relieve pain, and improve the patient's quality of life. Accordingly, methods of treatments contemplated to be used with the present invention may include: an intra-articular injection of a composition of the present invention followed by another intra-articular injection of another osteoarthritis treatment agent, examples of which include a viscosupplement, steroid, or other injectable osteoarthritis treatment agent; an intra-articular injection of a composition of the present invention followed by oral or intravenous administration of another osteoarthritis treatment agent such as a non-steroidal anti-inflammatory drug.

In some embodiments the invention may increase joint function and/or decrease pain, which therapeutic effects may be assessed indirectly or directly by using measures known to those skilled in the art to assess joint pain, joint function, weight-bearing, foot posture, extremity elevation time during walking, spontaneous mobility, and/or heat sensitivity.

Kits for use in treating osteoarthritis are also contemplated. An exemplary kit may include a calcium-channel blocker and a viscous carrier in one container, or each in separate containers. A needle may also be provided for ease of use. Optionally instructions for use are included within the kit. In some embodiments, the container is a syringe or vial.

The present invention may be illustrated by the following non-limiting examples:

### Example 1

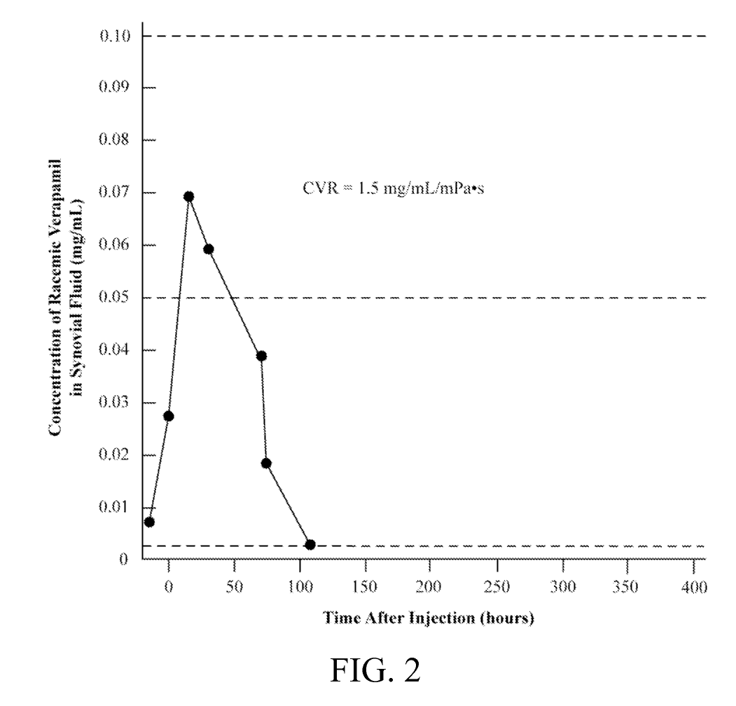

A test composition consisting of 15 mg of a racemic mixture of verapamil dissolved in 1 mL of a soluble biocompatible agent having a viscosity of 10 mPa·s (CVR=1.5 mg/mL/mPa·s) was placed where indicated in the apparatus depicted in FIG. 1, and the verapamil concentration in the simulated synovial fluid was measured at various times after placement by removing small aliquots of the synovial fluid and measuring the verapamil concentration therein using a spectrophotometric technique. The measurements were made periodically until the verapamil was cleared from the joint. A typical result is shown in FIG. 2. The highest concentration of verapamil in the synovial fluid. was about 0.069 mg/mL. The verapamil concentration remained above 0.05 mg/mL (indicated by the middle dotted line) for about 34 hours, and at least some verapamil remained in the simulated joint for about 100 hours (indicated by the lowest dotted line). During the 100-hour interval the average verapamil concentration was about 0.034 mg/mL.

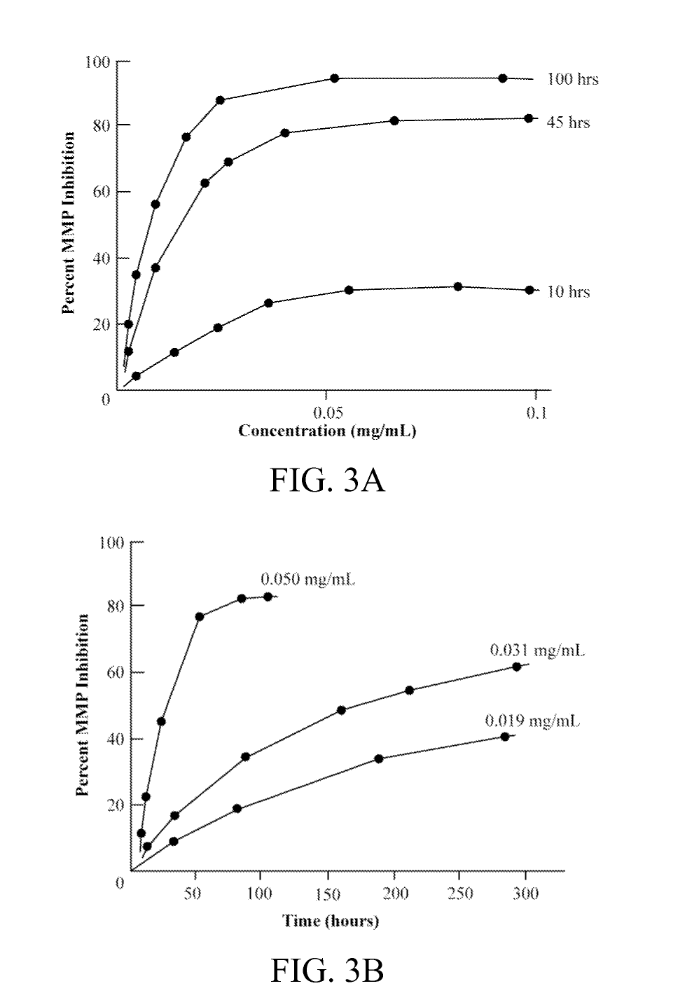

The data in FIGS. 3A and 3B show that the effect of racemic verapamil on inhibition of MMP activity secreted by synovial cells as functions of the verapamil concentration and the duration of exposure of the cells to the verapamil. FIG. 3A shows that when synovial cells are exposed to racemic verapamil for about 45 hours, MMP inhibition reaches its maximum value of about 81% at a verapamil concentration of about 0.05 mg/mL, and that further increases in the concentration do not result in enhanced MMP inhibition. When the exposure time is increased to 100 hours, a maximum MMP inhibition of 91% is observed. For shorter exposure times, the MMP inhibition is proportionately less. For example, at 10 hours, the maximum MMP inhibition is only 22%.

FIG. 3B depicts the effect on MMP inhibition for conditions of prolonged exposure of synovial cells to racemic verapamil. For example, when the verapamil concentration was 0.05 mg/mL, MMP inhibition of 81% was observed. For a verapamil concentration of 0.031 mg/mL, increased exposure time resulted in increased MMP inhibition up to about 300 hours, at which time the MMP inhibition was about 61%. For a verapamil concentration of 0.019 mg/mL the maximum MMP inhibition was 37% and occurred at 300 hours. Metabolic poisoning of synovial cells was observed for exposure times greater than about 100 hours at a verapamil concentration of 0.05 mg/mL, and after about 300 hours at 0.031 mg/mL and 0.019 mg/mL.

On the basis of data of the type depicted in FIGS. 3A and 3B, we found that exposure of synovial cells to 0.05 mg/mL of racemic verapamil for 34 hours produces a 61% reduction in MMP activity, and that exposure of said cells to 0.034 mg/mL for 100 hours produces a 31% reduction in MMP activity.

### Example 2

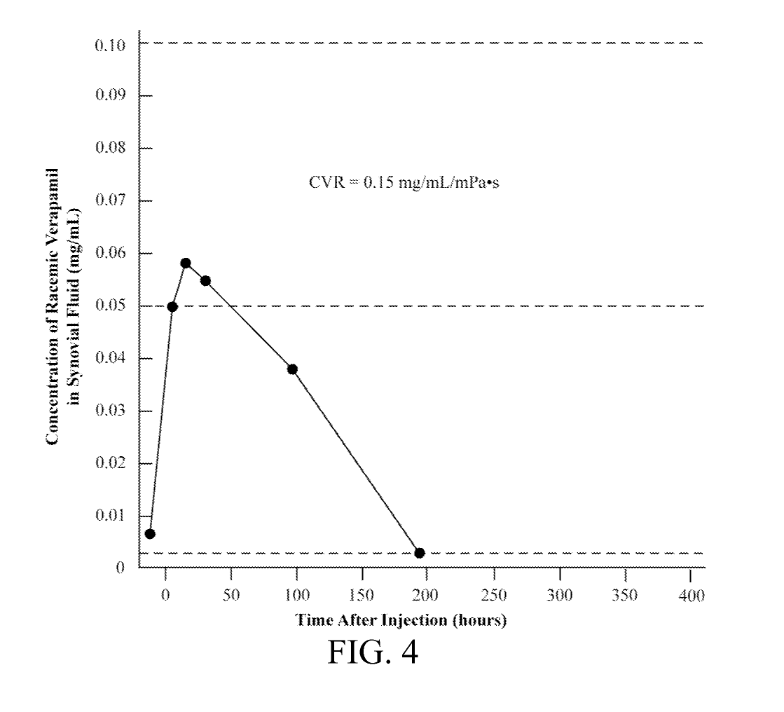

A test composition consisting of 15 mg of a racemic mixture of verapamil dissolved in 1 mL of a soluble biocompatible agent having a viscosity of 100 mPa·s (CVR=0.15 mg/mUmPa·s) was placed as indicated in the apparatus depicted in FIG. 1, and the verapamil concentration in the simulated synovial fluid was measured at various times after placement as described in Example 1. A typical result is shown in FIG. 4. The highest concentration of verapamil in the synovial fluid. was about 0.058 mg/mL, and the concentration remained above 0.05 mg/mL (indicated by the middle dotted line) for about 32 hours and at least some verapamil remained in the simulated joint for about 190 hours (indicated by the lowest dotted line). During the 190-hour interval the average verapamil concentration was about 0.024 mg/mL.

On the basis of data of the type depicted in FIGS. 3A and 3B, we found that exposure of synovial cells to 0.05 mg/mL of racemic verapamil for 32 hours produces a 59% reduction in MMP activity, and that exposure of said cells to 0.024 mg/mL for 194 hours produces a 27% reduction in MMP activity.

### Example 3

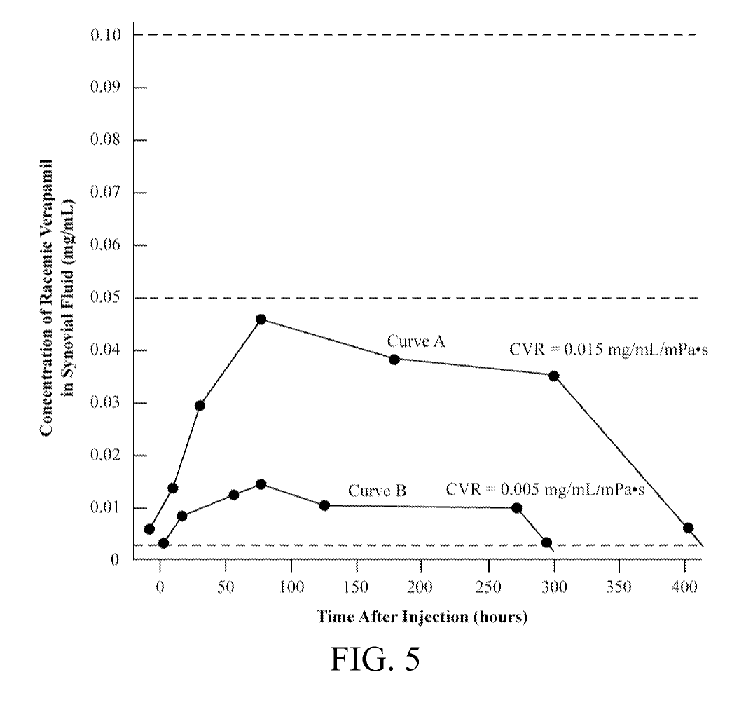

A test composition consisting of 15 mg of a racemic verapamil dissolved in 1 mL of a soluble biocompatible agent having a viscosity of 1000 mPa·s (CVR=0.015 mg/mL/mPa·s) was placed where indicated in the apparatus depicted in FIG. 1, and the verapamil concentration in the simulated synovial fluid was measured at various times after placement as described in Example 1. A typical result is shown in curve A in FIG. 5. The highest concentration of verapamil in the synovial fluid was about 0.046 mg/mL, and at least some verapamil remained in the simulated joint for more than 400 hours. When the amount of racemic verapamil was reduced to 5 mg, the verapamil was cleared from the joint within about 300 hours, as shown in curve B in FIG. 5; the average verapamil concentration during this period was 0.009 mg/mL.

On the basis of data of the type depicted in FIGS. 3A and 3B, we found that exposure of synovial cells to 0.046 mg/mL/mPa·s of racemic verapamil for 400 hours produces a reduction in MMP activity of about 63%. An unacceptably high number of synovial cells were metabolically poisoned as indicated by the high level of trypan blue staining. Exposure of cells to 0.009 mg/mL/mPa·s racemic verapamil for 300 hours produced a reduction of MMP activity of about 21%, but did not result in metabolic poisoning of synovial cells.

### Example 4

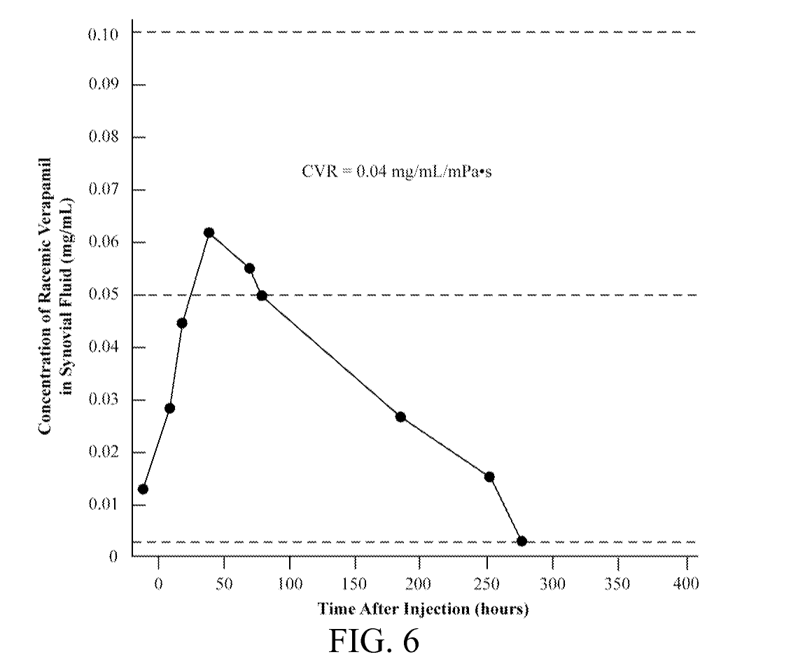

A test composition consisting of 20 mg of a racemic mixture of verapamil dissolved in 1 mL of a soluble biocompatible agent having a viscosity of 500 mPa·s (CVR=0.04 mg/mL/mPa·s) was placed where indicated in the apparatus depicted in FIG. 1, and the verapamil concentration in the simulated synovial fluid was measured at various times after placement as described in Example 1. A typical result is shown in FIG. 6. The highest concentration of verapamil in the synovial fluid. was about 0.062 mg/mL, and the concentration remained above 0.05 mg/mL (indicated by the middle dotted line) for about 62 hours, and at least some verapamil remained in the simulated joint for about 275 hours, and during this time interval the average verapamil concentration was about 0.025 mg/mL.

On the basis of data of the type depicted in FIGS. 3A and 3B, we found that exposure of synovial cells to 0.05 mg/mL of racemic verapamil for 62 hours produces an 87% reduction in MMP activity, and that exposure of said cells to 0.025 mg/mL for 275 hours produces a 46% reduction in MMP activity.

### Example 5

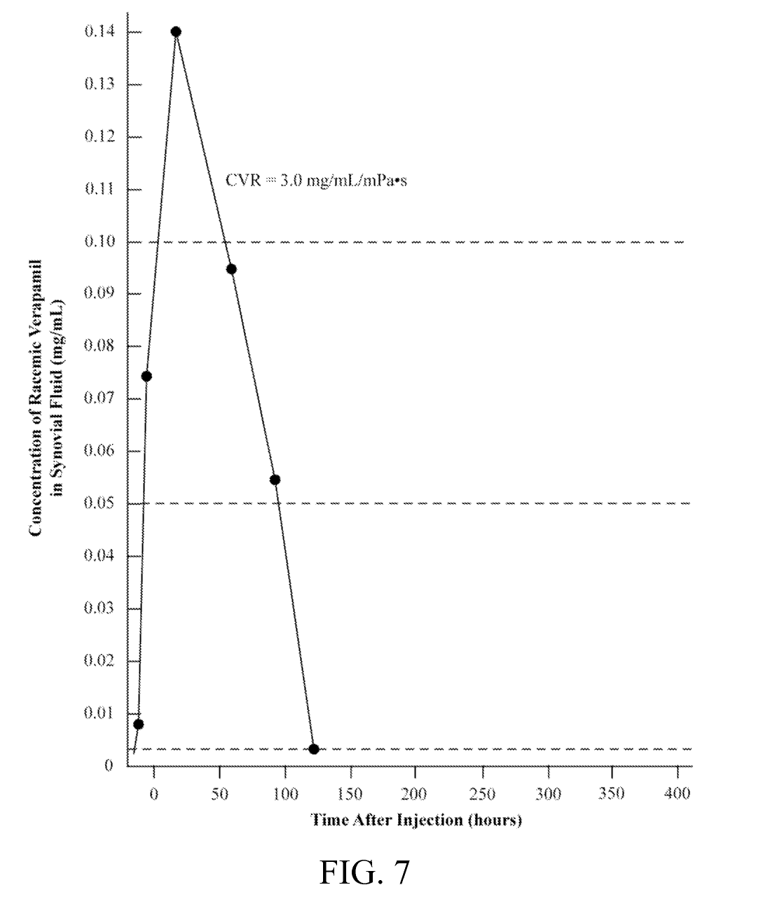

A test composition consisting of 30 mg of racemic verapamil dissolved in 1 mL of a soluble biocompatible agent having a viscosity of 10 mPa·s (CVR=3.0 mg/mL/mPa·s) was placed where indicated in the apparatus depicted in FIG. 1, and the verapamil concentration in the simulated synovial fluid was measured at various times after placement as described in Example 1. A typical result is shown in FIG. 7. The highest concentration of verapamil in the synovial fluid was about 0.140 mg/mL, and the concentration remained above 0.10 mg/mL (indicated by the top dotted line) for about 61 hours. On the basis of data of the type depicted in FIGS. 3A and 3B, we found that exposure of synovial cells to 0.10 mg/mL for 61 hours was lethal to the synovial cells, indicating that it was a nonfunctional embodiment of the invention.

### Example 6

Inflammatory arthritis is a cellular process known to those skilled in the art to simulate the cell signalling that occurs in osteoarthritis. Inflammatory arthritis was induced in BALB/c mice by mechanically injuring the knee joint and the effect of intra-articular injection of various compositions formulated in accordance with this invention on the disease process was studied using the method described in “Reduction of CpG-induced Arthritis by Suppressive Oligodeoxynucleotides,” A. Zeuner, K. J. Ishii, M. J. Lizak, I. Gursel, H. Yamada, D. M. Klinman, and D. Verthelyi; Arthritis & Rheumatism 46:2219-2224 (2002). One knee in each mouse was mechanically injured to create a condition of inflammatory arthritis, after which some mice were injected with a test composition and other mice were injected with the carrier alone (no calcium-channel blocker), and the injected joints were recovered and prepared for histological analysis.

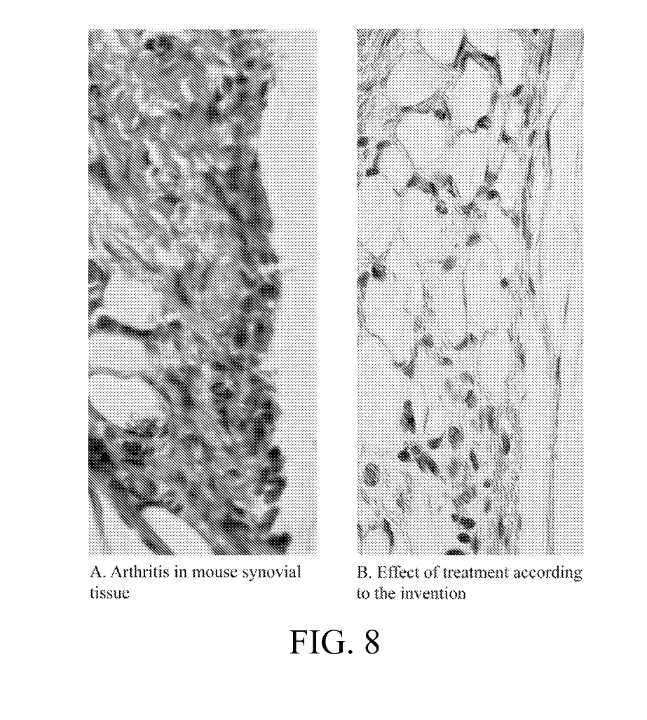

Inflammatory arthritis in response to the injury developed within 24 hours and peaked after about 3 days. The histologic changes included perivascular infiltration by mononuclear cells and hyperplasia of the synovial lining. The effect of various test compositions was studied. For example, the results obtained after injection of a composition containing the calcium-channel blocker nifedipine having a CVR of 0.028 mg/mL/mPa·s is shown in FIG. 8. The inflammatory response in a representative control mouse (carrier only) three days after injury is depicted in panel A, which shows a representative section of synovial tissue at a magnification of 400 times. The reduction in the hyperplasia of the synovial tissue in a mouse that received a test composition in accordance with the invention can be seen in panel B, which depicts a similar histological section. Similar results were obtained using racemic nifedipine in the CVR range 0.028-0.083 mg/mL/mPa·s and using racemic verapamil in the CVR range 0.06-0.36 mg/mL/mPa·s. The example shows that inflammatory arthritis can be effectively treated according to the invention.

### Example 7

Patient R.S. is a 43-year-old male with osteoarthritis of the right knee joint). An assessment of pain and, function of the joint was made immediately prior to treatment, and at various times after treatment, using the visual analog scale (VAS) for pain, and the Western Ontario and McMaster Universities (WOMAC) osteoarthritis index, which assesses pain, function and stiffness in arthritic joints. A more detailed description of the nature and use of these clinical endpoints is given in “Clinical Development Programs for Drugs, Devices, and Biological Products Intended for the Treatment of Osteoarthritis, U.S. Department of Health and Human services, Food and Drug Administration, July 1999”, which is incorporated by reference herein.

Immediately after the initial VAS and WOMAC measurements were made, the patient's right knee was injected with a total of 1.5 mg of a racemic mixture of verapamil in a carrier with a viscosity of about 1000 mPa·s. The injection procedure is described in “Viscosupplementation Under Fluoroscopic Control,” cited supra. The VAS and WOMAC scores obtained before and at various time points after the injection are shown in FIG. 8. Immediately before treatment, the patient had a VAS score as evaluated by a physician (“Physician VAS”) of 70, a VAS score as evaluated by the patient (“Patient VAS”) of 73, and a WOMAC score of 16. The patient was followed periodically up to 25 weeks and a reduction in pain and improvement in function was found. The example shows that temporal control of concentration of a calcium-channel blocker in joint synovial fluid achieved by means of controlling the static viscosity of the composition in accordance with the invention can produce a therapeutic effect in a patient suffering from osteoarthritis.

### Example 8

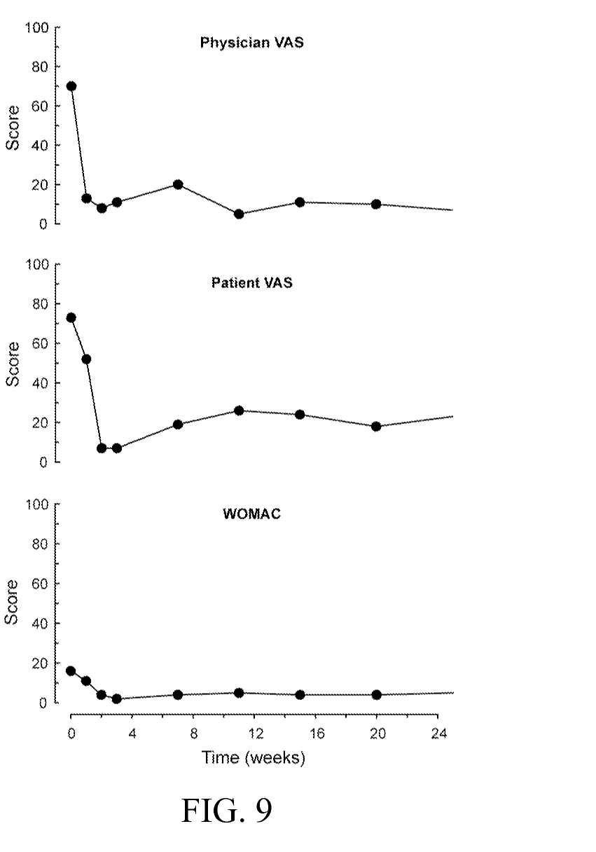

Patient T.J. is a 46-year-old male who suffered from osteoarthritis in the right knee joint. Prior to treatment, the patient had a physician VAS score of 47, a patient VAS score of 48 and a WOMAC score of 35. The patient's left knee was injected as described above in Example 6. The VAS and WOMAC scores obtained before and at various time points after the injection are shown in FIG. 9. Immediately before treatment, the patient had a VAS score as evaluated by a physician (“Physician VAS”) of 70, a VAS score as evaluated by the patient (“Patient VAS”) of 73, and a WOMAC score of 16. The patient was followed periodically up to 25 weeks and a reduction in pain and improvement in function was found. The example shows that temporal control of concentration of a calcium-channel blocker in joint synovial fluid achieved by means of controlling the static viscosity of the composition in accordance with the invention can produce a therapeutic effect in a patient suffering from osteoarthritis.

### Example 9

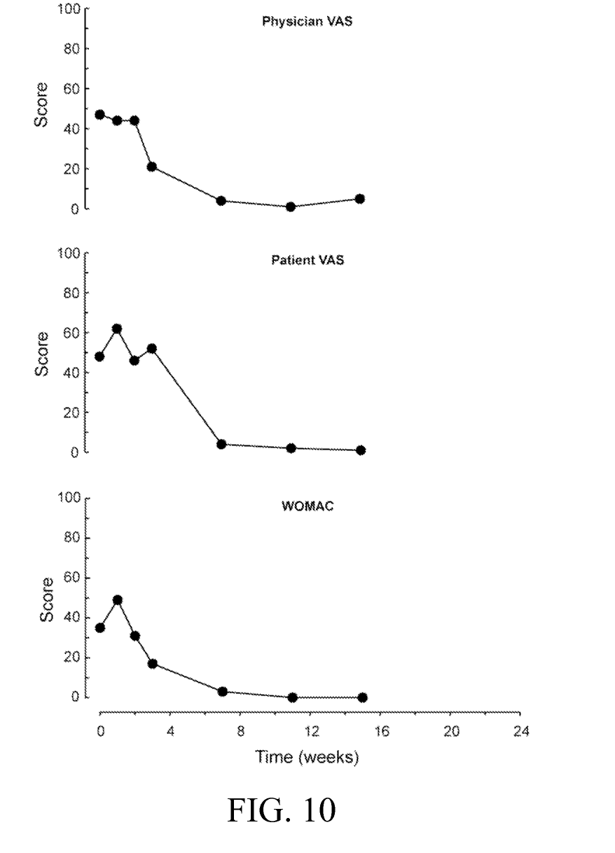

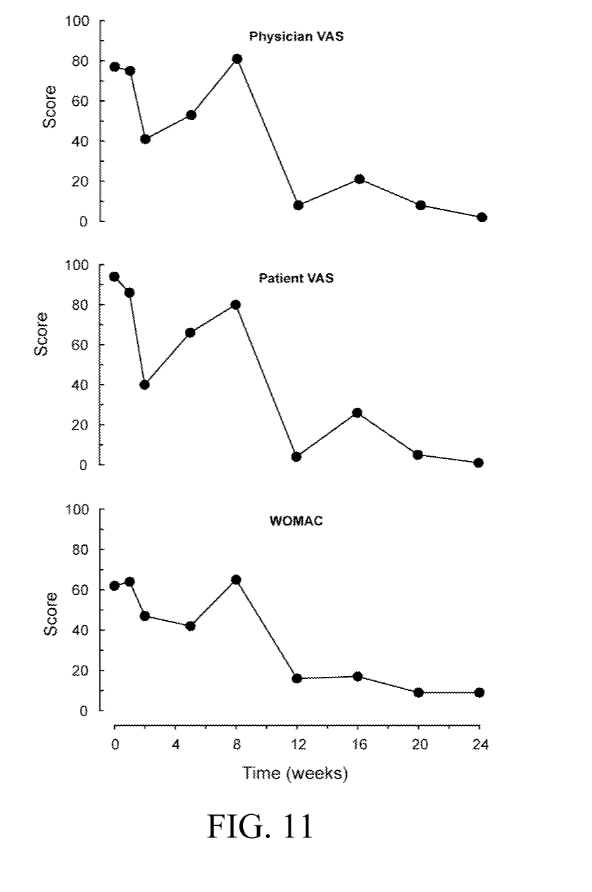

Patient C.E. is a 64-year-old male who suffered from osteoarthritis in the left knee joint. Prior to treatment, the patient had a physician VAS score of 77, a patient VAS score of 94 and a WOMAC score of 62. The patient's left knee was injected as described above in Example 6. The VAS and WOMAC scores obtained before and at various time points after the injection are shown in FIG. 10. Immediately before treatment, the patient had a VAS score as evaluated by a physician (“Physician VAS”) of 70, a VAS score as evaluated by the patient (“Patient VAS”) of 73, and a WOMAC score of 16. The patient was followed periodically up to 25 weeks and a reduction in pain and improvement in function was found. The example shows that temporal control of concentration of a calcium-channel blocker in joint synovial fluid achieved by means of controlling the static viscosity of the composition in accordance with the invention can produce a therapeutic effect in a patient suffering from osteoarthritis.

What is claimed is:

1. A composition suitable for intra-articular injection comprising:
(a) a calcium-channel blocker; and
(b) a soluble biocompatible carrier
wherein the ratio of the concentration of said blocker in the composition to the viscosity of said carrier is about 0.0001 to 2.0 mg/mL/mPa·s, whereby a therapeutically effective composition suitable for treating osteoarthritis will be provided.

2. The composition of claim 1, wherein said blocker is selected from the group consisting of verapamil, nifedipine, diltiazem, amlodipine, bepridil, felodipine, gallopamil, isradipine, nicardipine, nimodipine, and nitrendipine, and said carrier is selected from the group consisting of aqueous solutions containing glycerol, glycose, lactose, sorbitol, mannitol, polyethylene glycol having a molecular mass between 200-20,000 Daltons, ethyl oleate, ethyl laurate, collagen, gelatin, and hyaluronan.

3. A composition suitable for intra-articular injection comprising:
(a) a calcium-channel blocker; and
(b) a soluble biocompatible carrier
wherein the concentration of said in the composition blocker is about 2.0 mg/mL to about 30 mg/mL and the viscosity of said carrier is about 2 mPa·s to about 5000 mPa·s, whereby a therapeutically effective composition suitable for treating osteoarthritis will be provided.

4. The composition of claim 3, wherein said blocker is selected from the group consisting of verapamil, nifedipine, diltiazem, amlodipine, bepridil, felodipine, gallopamil, isradipine, nicardipine, nimodipine, and nitrendipine, and said carrier is selected from the group consisting of aqueous solutions containing glycerol, glycose, lactose, sorbitol, mannitol, polyethylene glycol having a molecular mass between 200-20,000 Daltons, ethyl oleate, ethyl laurate, collagen, gelatin, and hyaluronan.

5. A method for treating osteoarthritis which comprises injecting a calcium-channel blocker and a soluble biocompatible carrier into the closed cavity of an osteoarthritic joint wherein the ratio of the concentration of said blocker to the viscosity of said carrier is about 0.0001 to 2.0 mg/mL/mP·s.

6. A method for treating osteoarthritis which comprises injecting a calcium-channel blocker and a soluble biocompatible carrier into the closed cavity of an osteoarthritic joint wherein the concentration of said in the composition blocker is about 2.0 mg/mL to about 30 mg/mL and the viscosity of said carrier is about 2 mPa·s to about 5000 mPa·s, whereby a therapeutically effective composition suitable for treating osteoarthritis will be provided.
